(19) United States
(12) Patent Application Publication
(10) Pub. No.: US 2025/0040861 A1
(43) Pub. Date: Feb. 6, 2025

(54) DEVICE FOR DETECTING BRAIN ACTIVITY OF A MAMMAL AND ASSOCIATED METHOD

(71) Applicant: NAOX TECHNOLOGIES, Palaiseau (FR)

(72) Inventors: Emmanuel LANGE, Palaiseau (FR); Hugo DINH, Palaiseau (FR); Khalil KABABE, Palaiseau (FR); Mario VALDERRAMA, Palaiseau (FR)

(73) Assignee: NAOX TECHNOLOGIES, Palaiseau (FR)

(21) Appl. No.: 18/785,019

(22) Filed: Jul. 26, 2024

(30) Foreign Application Priority Data
Aug. 1, 2023 (EP) 23 306 321.3

(51) Int. Cl. A61B 5/372; A61B 5/00; A61B 5/16; A61B 5/256; A61B 5/291; A61B 5/31

(52) U.S. Cl. CPC A61B 5/372; A61B 5/165; A61B 5/256; A61B 5/291; A61B 5/31; A61B 5/6817; A61B 5/7221; A61B 5/7225; A61B 5/7246; A61B 5/7264; A61B 5/7405; A61B 2560/0443; A61B 2560/0468; A61B 2562/04; A61B 2562/182; A61B 2562/227

(57) ABSTRACT

A device for brain activity of a mammal, the device having at least one earpiece including: a main body having an elongation portion extending along an elongation axis; and an earmold configured to be inserted into an ear canal of the mammal and having at least one electrode assembly, the electrode assembly including one measurement electrode arranged on an outer surface of the earmold and configured to deliver an electrical signal representative of the brain activity; a processing unit, wherein the electrode assembly includes at least one preamplifier positioned upstream of the processing unit in the direction of propagation of the electrical signal.

### FIELD

The present invention relates to the field of devices for detecting brain activity of a mammal. The invention especially applies in determining a physiological or psychological state of a mammal based on the detected brain activity, and relates in particular to methods and devices for determining such a state by measuring an electroencephalogram (EEG) signal.

### BACKGROUND

An in-ear device for measuring biological data of a human, such as the one described by US 2018/0235540, is known, which comprises an interchangeable portion intended to be inserted into an ear canal provided on its outer surface with electrodes intended to detect an electrical signal from the heart.

The detection of electrical signals by the electrodes may lack accuracy. Indeed, such an in-ear device is sensitive to external and internal noises.

The invention aims at providing a solution in this regard and, more particularly, at overcoming the aforementioned disadvantages and at providing a biological data measuring device capable of providing more accurate signals, and having a simple, inexpensive and compact structure.

### SUMMARY

This invention thus relates to a device for detecting brain activity of a mammal, the device comprising at least one earpiece configured to be worn on an ear of the mammal, the earpiece comprising:
a main body comprising a housing portion and an elongation portion which extends along an elongation axis from the housing portion, the elongation portion being configured to be inserted into an ear canal of the ear of the mammal, and
an earmold configured to be inserted into an ear canal of the ear of the mammal, the earmold having a channel extending along a earmold axis, and a skirt surrounding the channel, the channel being adapted to receive said elongation portion to removably mount the earmold onto the main body, at least the skirt of the earmold being made of an elastically deformable material, said earmold comprising at least one electrode assembly, the electrode assembly comprising a measurement electrode arranged on an outer surface of the skirt of the earmold to contact the ear canal and configured to deliver an electrical signal representative of the brain activity of the mammal, said electrical signal having an electrical intensity;
a processing unit arranged in the housing portion of the main body, the earpiece comprising at least one first electrical track arranged in the channel of the earmold and connected to the measurement electrode, and at least one second electrical track arranged in the elongation portion of the main body and configured to convey the electrical signal delivered by the measurement electrode to the processing unit,
   wherein said electrode assembly comprises a preamplifier having an intensity gain adapted to adapt the electrical intensity of the electrical signal delivered by the measurement electrode (42ₖ) of said electrode assembly, the preamplifier being positioned upstream of the processing unit in a direction of propagation of the electrical signal delivered by the measurement electrode of said electrode assembly, so that the processing unit is configured to process the electrical signal adapted in intensity from the electrode assembly.

Thus, the preamplifier allows amplifying the electrical intensity of the electrical signals delivered by the measurement electrode of the device, making the electrical signals more prominent with respect to external and internal noises also measured by the device when the device is in use.

The preamplifier may be adapted to amplify the intensity of the electrical signal detected by the measurement electrode of the electrode assembly.

In some embodiments, the preamplifier of the electrode assembly is housed in the elongation portion.

In some embodiments, the preamplifier presents a working frequency range extending from 0.1 Hz to 1 kHz.

Thus, this position of the at least one preamplifier allows amplifying the electrical signals detected by the measurement electrode close to the location where those electrical signals are captured, therefore minimizing the capturing of unwanted signals.

In some embodiments, the preamplifier is coupled to an ESD (electrostatic discharge) circuit. Therefore, advantageously, the ESD circuit protects the device according to the invention from electrostatic discharges that can occur for instance when a user of the device touches the device.

In some embodiments, the preamplifier comprises a follower circuit. A follower circuit is also referred to as buffer or unity gain amplifier and is based on an operational amplifier. Advantageously, the output provided by the operational amplifier is identical to the input in terms of voltage, but with a higher current capability. Therefore, advantageously, the follower circuit provide a low output impedance to minimize signal losses when the follower circuit is connected to a load.

In some embodiments, the preamplifier comprises an impedance matching circuit. Such an impedance matching circuit modifies the impedance of an electrical signal source, here, the body of the mammal, to match the impedance of a connected load (here, processing unit that will be further described). In this way, the impedance matching circuit optimizes the power transfer between the body of the mammal and the load by minimizing losses due to impedance mismatch. Typically, when the mammal is a human person, the input impedance lies in the Tera-ohms (T (2) range, whereas, at the output of the impedance matching circuit, the impedance lies in the kilohms (k (2) range.

In some embodiments, the preamplifier is mounted on a printed circuit board of maximum dimension 3 millimeters.

In some embodiments, the printed circuit board is made out of a flexible material, so as to be folded. In this manner, the at least one preamplifier can be integrated for instance inside the elongation portion of the main body in an easy and compact manner.

For instance, the flexible material can be chosen among polyimides such as Kapton, polyetheretherketone (PEEK), polymers, PTFE (Teflon).

In some embodiments, the printed circuit board comprises a U-shape so as to embrace additional components embedded in the elongation portion. In this manner also, the at least one preamplifier can be integrated for instance inside the elongation portion of the main body in an easy and compact manner.

In some embodiments, the earpiece is further configured to transmit a sound signal into the ear canal by means of at least one electroacoustic transducer housed in the elongation portion, the preamplifier being attached to the electroacoustic transducer along a circumferential direction of the elongation axis. Therefore, the device according to the invention can be multifunctional while remaining compact.

In some embodiments, the elongation portion of the main body embeds a signaling LED and the at least one preamplifier is mounted on the signaling LED.

The earmold may be arranged to be rotatable about the elongation axis on the elongation portion so as to allow the measurement electrode to be directed to an area of the brain of said mammal, the second electrical track extending in a circumferential direction around the elongation axis on at least a portion of an outer surface of the elongation portion, the first electrical track presenting an end portion extending radially with respect to the elongation axis to be in contact with the second electrical track.

The housing portion may be configured to be received in the ear conch of the ear of the mammal.

Said at least one electrode assembly may include a plurality of electrode assemblies each comprising one measurement electrode and one preamplifier.

Said plurality of electrode assemblies may comprise at least first and second electrode assemblies, at least a first measurement electrode of the first electrode assembly being configured to deliver a first electrical signal and a second measurement electrode of the second electrode assembly being configured to detect a second electrical signal, said at least first electrical track comprises at least two first electrical tracks, and said at least second electrical track comprises at least two second electrical tracks, said at least two first electrical tracks being electrically insulated from each other, said at least two second electrical tracks being electrically insulated from each other, the processing unit being configured to detect brain activity as a function of said first electrical signal delivered by said first measurement electrode and adapted in intensity by the first preamplifier of the first electrode assembly and of said second electrical signal delivered by said second measurement electrode and adapted in intensity by the second preamplifier of the second electrode assembly, in particular as a function of a difference between said first electrical signal and said second electrical signal

In some embodiments referred to as single earpiece embodiments, the first and second electrode assemblies are arranged on a single earpiece, the first and second measurement electrodes being spaced apart along the earmold axis, the two first electrical tracks being spaced apart along the earmold axis and the two second electrical tracks being spaced apart along the elongation axis. Those single earpiece embodiments are based on the use of a device using only one earpiece.

In other embodiments referred to as dual earpiece embodiments, the device comprises two earpieces configured to be arranged respectively in a first ear canal of the mammal and a second ear canal of the mammal and configured to convey respective electrical signals to the processing unit, and the first and second electrode assemblies are arranged respectively on the two earpieces. Those dual earpiece embodiments are based on the use of a device using two earpieces.

The processing unit may further be configured to determine physiological or psychological state based on the detected brain activity

Another aspect of the invention relates to computer-implemented method for determining a physiological or psychological state of a mammal by means of the device previously described, the method comprising:
determining an electroencephalogram signal of the mammal based on the delivered electrical signals,
determining an amplitude of at least one brain wave in a predefined frequency range as a function of said electroencephalogram signal, and
determining a psychic state based on said amplitude of at least one brain wave by comparing said amplitude to a predetermined threshold.

In some embodiments, the method further comprises a step of computing a quality index of the electrical signals delivered by the measurement electrodes based on a correlation computation between a common mode measurement and said difference between said first electrical signal and said second electrical signal or said difference between said two electrical signals.

### Definitions

In the present invention, the following terms have the following meanings:

The terms “adapted” and “configured” are used in the present disclosure as broadly encompassing initial configuration, later adaptation or complementation of the present device, or any combination thereof alike, whether effected through material or software means (including firmware).

The term “reference electrode” refers to an electrode used in EEG to establish a baseline voltage or reference point for measuring the electrical potentials recorded by the measurement electrodes. It serves as a point of comparison against which the electrical activity from other electrodes is measured. The reference electrode does not directly measure brain activity; instead, it provides a fixed voltage level that helps in interpreting the electrical signals from other electrodes.

The term “measurement electrode”, also known as a measure electrode or recording electrode, refers to an electrode configured to directly measure the electrical activity generated by the brain. It detects the voltage fluctuations resulting from the electrical signals produced by neural activity in the brain. The measure electrodes are the primary electrodes used for capturing EEG signals.

### BRIEF DESCRIPTION OF THE DRAWINGS

FIG. 1 represents a side view of a device for detecting brain activity of a mammal according to some embodiments.

FIG. 2 represents an inside view of the device of FIG. 1 in the plane of FIG. 1.

FIGS. 3A and 3B are three-dimensional perspective views of a printed circuit board embedding a preamplifier used in the device of FIGS. 1 and 2, in an unfolded configuration (3A) and in a folded configuration while embedded (3B) in a channel, according to some embodiments.

FIG. 4 is a schematic figure of processing unit configured to be used in a device as represented in FIGS. 1 and 2.

FIG. 5 represents a device for detecting brain activity of a mammal according to some embodiments different from FIGS. 1 and 2.

FIG. 6 represents an example of a flow chart representing steps for implementing a method for determining a physiological or psychological state of a mammal with a device such as illustrated on FIGS. 1, 2 and 5.

### DETAILED DESCRIPTION

This invention relates to a device 1a, 1b for detecting brain activity of a mammal, the device comprising at least one earpiece 2, 2′ and to a computer-implemented method 100 for determining a physiological or psychological state of the mammal by means of at least one processor of the device 1. Preferably, the mammal is a person.

One single earpiece 2 configured to be worn on an ear of the mammal and comprising at least two measurement electrodes can be used. Alternatively, two earpieces 2, 2′, configured to be configured to be worn on respective ears of the mammal, each earpiece comprising at least one measurement electrode, can be used, both earpieces 2, 2′ being possibly identical or different from each other. It will be further explained and described how the computer-implemented method 100 relies on the use of at least two measurement electrodes, both measurement electrodes being comprised into either one earpiece 2 or each of the two electrodes being comprised into one among two separate earpieces 2, 2′.

More specifically, the device 1a, 1b is configured for determining electroencephalogram (EEG) data of a person as brain activity.

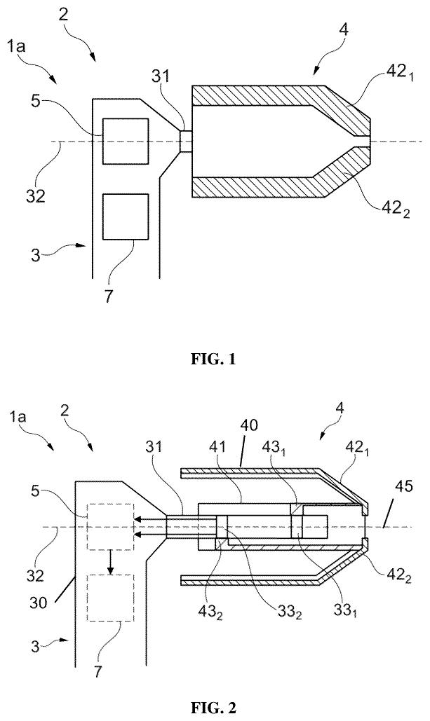

The earpiece 2, 2′ will now be described and FIG. 1 illustrates an example of the earpiece 2, 2′. For example, the earpiece 2, 2′ is shaped and sized like an earphone, and includes a main body 3 and a removable earmold 4.

The main body 3 comprises a housing portion 30 and an elongation portion 31 which extends along an elongation axis 32 from the housing portion 30. In one example, the elongation portion 31 may be a body of revolution, wherein the elongation axis is an axis of revolution. The housing portion 30 may be configured to be received in the ear conch of the ear of the person while the elongation portion 31 is configured to be inserted into the ear canal 10, 10′ of the ear of the person.

The earmold 4 is configured to be fitted on the elongation portion 31 of the main body 3 and inserted into an ear canal 10, 10′ of the person. The earmold 4 comprises a channel 41 extending along a earmold axis 45, and a skirt 40 surrounding the channel 41. The channel 41 is adapted to receive the elongation portion 31 of the main body 3 to removably mount the earmold 4 onto the main body 3. At least the skirt 40 of the earmold 4, and possibly all the earmold 4, is made of an elastically deformable material. The earmold 4 comprises a plurality of N electrode assemblies. Each electrode assembly comprises a measurement electrode (42₁, 42ₖ . . . 42_{N}) arranged on an outer surface of the skirt 40 of the earmold 4 to contact the ear canal 10, 10′. For instance, the measurement electrodes (42₁, 42ₖ . . . 42_{N}) extend along the entire length of the earmold 4 in the direction of the earmold axis 45. For instance, the measurement electrodes are spaced apart in a circumferential direction about the earmold axis 45. Other arrangements of the measurement electrodes could be possible. On FIG. 1, two measurement electrodes 42₁ and 42₂ are visible.

Each measurement electrode 42ₖ is configured to deliver an electrical signal representative of the brain activity of the mammal. The earmold 4 can arranged to be rotatable about the elongation axis 32 so as to allow at least one of the measurement electrodes 42ₖ to be directed to an area of the brain of the person. For instance, the earmold 4 is made of insulating material, e.g., silicone, and the measurement electrodes 42ₖ are, for example, pieces of conductive fabric embedded in the silicone of the earmold 4, e.g., by gluing. The skirt 40 of the earmold 4 is sized so as to ensure contact between the wall of the ear canal of the person and the measurement electrodes 42ₖ.

As illustrated on FIG. 2, the earpiece 2, 2′ comprises first electrical tracks 43ₖ electrically insulated from each other arranged in the channel 41 of the earmold 4 and connected to the measurement electrodes 42ₖ. For instance, each first electrical track 43ₖ can comprise an end portion in the form of a contact pin extending radially with respect to the elongation axis 32 and connected to the measurement electrodes 42ₖ by means of electrically insulated electrical wires, the contact pins being embedded on an inner surface of the channel 41 of the earmold 4.

The housing portion 30 of main body 3 accommodates a processing unit 5 configured to receive the electrical signals picked up by the measurement electrodes 42ₖ of the earmold 4, process these signals and transform them into digital signals. Accordingly, the earpiece 2, 2′ comprises second electrical tracks 33ₖ electrically insulated from each other, arranged in the elongation portion 31 of the main body 3 and configured to convey the electrical signals delivered by the measurement electrodes 42ₖ to the processing unit 5. Each second electrical track 33ₖ may extend in a circumferential direction around the elongation axis 32 on at least a portion of the outer surface of the elongation portion 31 to be in contact with the first electrical track 43ₖ. For instance, as illustrated on FIG. 2, the second electrical tracks 33ₖ are annular electrical tracks having the elongation axis 32 as their axis and arranged at a distance from each other in the direction of the elongation axis 32. Each of the annular tracks is connected to a measurement electrode 42ₖ of the earmold 4 by one corresponding first electrical track 43ₖ. The contact pins can be arranged opposite to the annular electrical tracks so as to maintain an electrical contact.

In some embodiments, the elongation portion 31 is made of insulating material and the second electrical tracks 33; are formed by a metal deposit on the elongation portion 31. The second electrical tracks 33ₖ are connected to the processing unit 5 by electrical wires arranged within the elongation portion 31.

Each electrode assembly includes preamplifier 6, also referred to as buffer, positioned upstream of the processing unit 5 in a direction of propagation of the electrical signals delivered by the measurement electrodes 42ₖ. There are N preamplifiers 6, each among them configured to receive a corresponding electrical signal from one among the N measurement electrodes 42ₖ.

The preamplifier 6 of each electrode assembly has an intensity gain adapted to adapt, and especially amplify, an electrical intensity of the electrical signal delivered by the measurement electrode of the same electrode assembly. The processing unit 5 thus receives and processes the electrical signal adapted, especially amplified, in intensity from each of the electrode assemblies.

Each of the N preamplifiers 6 is configured to perform impedance matching. In other words, the impedance at the input of one preamplifier 6 is higher than the impedance at the output of this preamplifier. For instance, each among the N preamplifiers 6 comprises a follower circuit based on a differential amplifier.

Advantageously, the intensity gain of each among the N preamplifiers 6 is adjustable, therefore allowing versability in the distribution of intensity gains allowed by the N preamplifiers. In other words, the electrical intensity of some electrical signals among the electrical signals delivered by the measurement electrodes 42 may be amplified in a stronger manner than the electrical intensity of the other electrical signals, so as to emphasize differential measurements between the electrical signal with strongly amplified electrical intensity (i.e. signal representative of the neural or brain activity obtained from an electrode working as measurement electrode) and an electrical signal with lower electrical intensity from another electrode (i.e., baseline voltage obtained from an electrode working as reference electrode). As will be explained below, the device 1a, 1b is configured to perform and process such differential measurements. The adjustability of the intensity gain in an independent way of one or more of N preamplifiers 6 thus advantageously allows increasing the signal to noise ratios of the different delivered electrical signals. In other words, as the neural recordings are performed using differential measurements, the possibility of strongly amplify the electrical signal(s) of the measurement electrodes using these N preamplifiers 6 increases the noise immunity of the device 1a, 1b and allows to recover a neural signal with an improved dynamic, i.e., a neural signal carrying more information. Meanwhile, the N preamplifiers 6 render the electrical intensity of the electrical signals delivered by the measurement electrodes 42ₖ adjustable, therefore allowing possibly reducing the power consumption of the device 1a, 1b. This embodiment allowing to directly amplify analogically the desired electrical signal(s) provides better signal-to-noise ratio, while also reducing the energy consumption, with respect to other alternative methods which improves the difference between the baseline voltage and the measurement signal by numerically applying weighting factors to the electrical signals during signal processing.

The presence of the preamplifiers 6 upstream the processing unit 5 is also advantageous for the following reasons. The electrical intensity of the electrical signals corresponding to biological signals from the person and detected by the measurement electrodes 42ₖ is of the order of magnitude of picoamps, which is comparable to the order of magnitude of external noises generated by external induction loops (with for example the main power supply) and internal noises also detected by the measurement electrodes. By internal noises, it is meant noises created by sources of noise present in the body of the mammal. Such sources of noise are for instance generated from the contraction of the facial muscles, eyelids or eyes movements, etc.

Therefore, the preamplifiers 6 allow preserving the input voltage of the electrical signals delivered by the electrodes 42ₖ while increasing the electrical intensity. Typically, the impedance at the input of the preamplifiers 6 roughly corresponds to the impedance of the mammal skin and lies in the mega-ohm range, while the impedance at the output of the preamplifiers 6 lies in the kilohm range. Indeed, the brain of the mammal can be modeled as a voltage generator with an internal impedance. The internal impedance comprises the resistance of the head of the mammal and the resistance of the skin of the mammal.

Another advantage is the proximity of the preamplifiers 6 to the second electrical tracks 33ₖ, since the preamplifiers 6 are located in the elongation portion 31 of the main body 3. This allows to amplify the corresponding electrical signals close to the location where the electrical signals of interest are captured and as a consequence to minimize the capturing of unwanted signals.

Besides, the presence of the intensity gain tunable preamplifiers 6 alleviates the need for downstream digital amplification, which contributes to power consumption reduction.

In some embodiments, each of the N preamplifiers 6 is coupled to an ESD protection circuit.

In some preferred embodiments, the size of each of the N preamplifiers 6 is configured so that the earpiece 2, 2′ can be used when the person is a child. For instance, the size of the preamplifier/earpiece is smaller than a few millimeters, for instance smaller than 3 mm.

Advantageously, the N preamplifiers 6 are mounted on a printed circuit board 61.

In some preferred embodiments, the printed circuit board 61 is flexible, so as to be folded. For instance, the printed circuit board can made of a polyimide such as Kapton, polyetheretherketone (PEEK), polymers, PTFE (Teflon).

In alternative embodiments, the printed circuit board presents a U-shape or C-shape or may be fold into a U-shape or a C-shape, so as to embrace other components embedded in the elongation portion 31 of the main body 3.

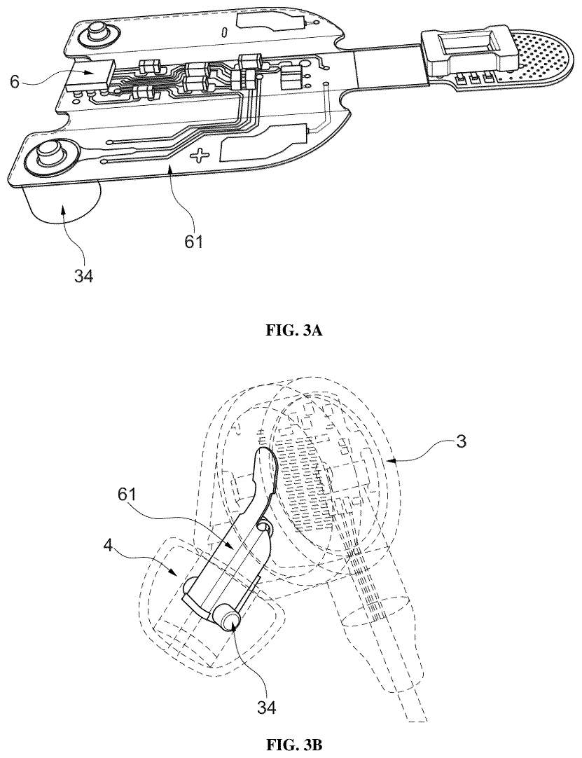

FIGS. 3A and 3B are examples of three-dimensional perspective views of one among the N preamplifiers 6 mounted on a printed circuit board. An electrode 34 is visible on FIG. 3A, which is meant to be connected to one of the first tracks 43ₖ of the earmold 4. FIG. 3B shows an example of the printed circuit board 61 on which one among the N preamplifiers 6 is mounted, and in a folded configuration so as to be integrated within the device 1. The corresponding electrode 34 is visible, as well as the earmold 4. As the earmold 4 is made out of a flexible material, it is pressed when the device 1 is inserted into one ear canal of the mammal for use. The distance between the ear canal surface and the end of the electrode 34, in this use configuration, is less than 1.5 mm.

For instance, in some embodiments, the earpiece 2, 2′ is further configured to transmit a sound signal into the ear canal by means of at least one electroacoustic transducer housed in the elongation portion 31 of the main body 3. In those embodiments, the printed circuit board in U-shape or C-shape, on which the preamplifiers 6 are mounted, can be positioned around said at least one electroacoustic transducer and, optionally, attached to the at least one electroacoustic transducer, for instance by gluing.

In other embodiments, the earpiece 2, 2′ can comprise an additional functional component that can be positioned within the elongation portion 31 of the main body 3, such as a signaling LED. Similarly, in those embodiments, the printed circuit board on which the preamplifiers 6 are mounted can be attached to the additional functional component.

In this manner, the inclusion of the preamplifiers 6 in the earpiece 2, 2′ can be carried out simply while keeping the earpiece 2, 2′ compact in size. Other arrangements of the preamplifiers 6 inside the elongation portion 31 are possible. The positioning of the printed circuit board (i.e., the preamplifiers 6) in the elongation portion 31 is advantageous as it allows to bring the amplifiers 6 closer to the measurement electrodes 42ₖ so as to reduce the parasite noise.

The processing unit 5 is configured to output processed signals by digitizing and processing the electrical signals delivered by the measurement electrodes 42ₖ and that have passed through the preamplifiers 6, towards at least one processor 7 that will be described further and that are configured to implement the computer-implemented method 100 for detecting the brain activity and possibly determining a physiological or psychological state of the mammal.

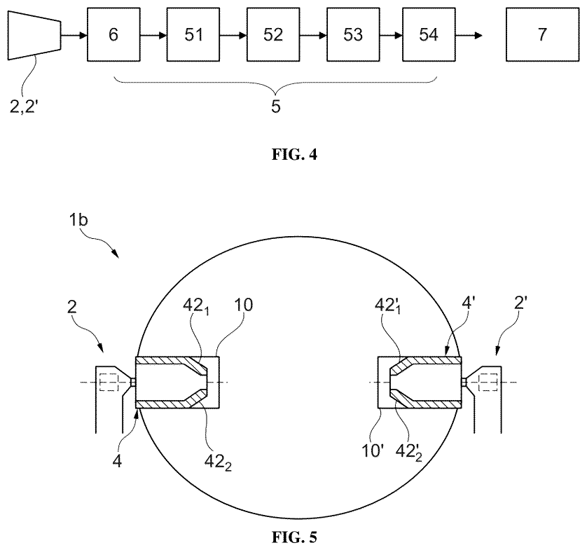

An example of chain of components comprised in the processing unit 5 including the preamplifiers 6 and other components downstream of the preamplifiers 6 is illustrated on FIG. 4 and will be described below. It is to be noted that the preamplifiers 6 present, as explained above, a specific and independent technical function, and associated advantages, among the chain of components comprised in the processing unit 5.

Advantageously, the processing unit 5 is manufactured in a compact manner and can be encompassed in a limited volume. For instance, when the processing unit 5 is embedded inside the main body 3, it can be encompassed inside a cylinder of radius 6 mm and thickness 1 mm, leading to an encompassing volume of approximately 200 mm³.

First, an amplifier 51 is arranged to amplify the amplitude of the or each electrical signal. The gain of the amplifier 51 may be 10000 times in a single or multiple stages. The amplifier 51 is connected to an analog-to-digital converter (ADC) 52 configured to digitize the or each electrical signal. The ADC 52 is connected to a digital signal processing module 53 configured to attenuate any spurious signals picked up by the electrodes. These spurious signals may be due to the movement of the person's head, a movement of the electrical wires connected to the device 1, or the environment of the device 1. This digital signal processing module 53 includes in particular a bandpass filter, but also signal processing functions, in particular a function allowing a low frequency (or constant) component of the signal to be removed. This low frequency (or constant) component is present due to the fact of choosing a reference electrode inside the ear canal without using a mass located outside the ear canal. The bandwidth of the bandpass filter is further configured to select electrical signals from a predetermined organ, in particular the brain. For example, the bandwidth of the bandpass filter is between 0.5 Hz and 60 Hz, in particular between 1 Hz and 40 Hz. The bandpass filter is connected to a communication module 54 configured to transmit the signals digitized by the ADC 52 and filtered by the bandpass filter to the processor 7, in a wired or wireless manner.

The processor 7 comprises at least one processor. The processor 7 may be integrated into the main body 3 or separate therefrom, to determine EEG data based on the processed signals received from the processing unit 5.

In other embodiments, the processing unit 5 may also be arranged outside the main body. In those embodiments, the device 1a, 1b may comprise an antenna in the main body 3 or in the elongation portion 31 downstream of the at least one preamplifier 6 so as to transmit the electrical signals captured by the measurement electrodes to the processing unit 5.

As mentioned before, the device 1a, 1b may comprise one earpiece 2 (the device is then referred to as 1a) or two earpieces 2, 2′ (the device is then referred to as 1b).

In so-called single earpiece embodiments, where the device is referred to as device 1a, one earpiece 2 configured to be inserted into an ear canal of the mammal and comprising at least two measurement electrodes is used. The earmold 4 of the earpiece 2 comprises at least a first measurement electrode 42₁ and a second measurement electrode 42₂ comprised among the measurement electrodes 42ₖ, each among the first measurement electrode 42₁ and the second measurement electrode 42₂ being configured to deliver respectively a first electrical signal and a second electrical signal. In those embodiments, the at least one processor of the processor 7 is configured to determine a neural signal as a function of the first electrical signal and of the second electrical signal, in particular as a function of a difference between the first electrical signal and the second electrical signal. More specifically, one among the first measurement electrode 42₁ and the second measurement electrode 42₂ is chosen as the reference electrode, relative to which the potential will be calculated and the other electrode is therefore chosen as measurement electrode. The brain potential is calculated by differentiating the signals picked up by the other electrode from the signal picked up by the reference electrode. Preferably, the first measurement electrode 42₁ and the second measurement electrode 42₂ are as identical as possible.

In alternative so-called dual earpiece embodiments, where the device is referred to as device 1b, two earpieces 2, 2′, configured to be inserted into each ear canal of the mammal are used, as represented on FIG. 5. Each earpiece 2, 2′ comprises at least one measurement electrode. In those embodiments, the at least one processor of the processor 7 is configured to determine the physiological or psychological state as a function of a signal measured by an electrode of the earpiece 2 hosted in the first ear canal and a signal measured by an electrode of the earpiece 2′ hosted in the second ear canal, in particular as a function of a difference between both signals. Similarly to the single earpiece embodiments, one electrode of one earpiece 2, 2′ is chosen as the reference electrode (while the other electrode will act as the measurement electrode), relative to which the potential will be calculated. The brain potential is calculated by differentiating the signals picked up by one electrode of the other earpiece from the signal picked up by the reference electrode.

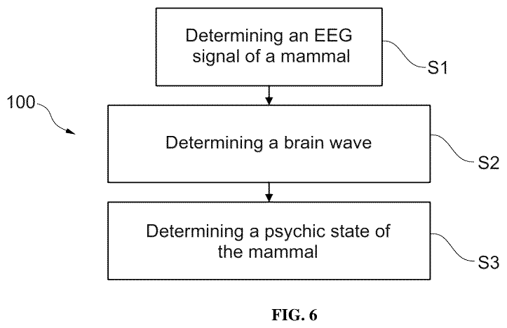

Another aspect of the invention relates to the computer-implemented method 100 for determining a physiological or psychological state of the mammal. The computer-implemented method 100 can be implemented by the processor 7 of the device 1a, 1b previously described. FIG. 6 represents an example of steps that may be perform to implement the method 100.

In a step S1, the at least one processor determines an electroencephalogram signal of the mammal based on the detected electrical signals. As previously evoked in the description of the device 1a, 1b, the at least one processor computes the brain potential based on the difference between the signals of two electrodes, either two electrodes from one single earpiece 2, or one electrode of one earpiece 2 and another electrode of another earpiece 2′.

In a step S2, the at least one processor determines an amplitude of at least one brain wave in a predefined frequency range as a function of the electroencephalogram signal. For instance, a Fourier transform can be applied to determine the frequencies contained in the electroencephalogram signal. For instance, if the signal frequency is between 5 Hz and 15 Hz, in particular 10 Hz, the brain wave is of the α type, and if the signal frequency is between 15 Hz and 25 Hz, in particular 20 Hz, the brain wave is of the β type.

In a step S3, the at least one processor determines a psychic state based on the amplitude of at least one brain wave by comparing the amplitude to a predetermined threshold. For instance, a level of attention of the person can be determined based on the comparison.

Optionally, the at least one processor can compute a quality index of the electrical signals detected by the electrodes based on a correlation computation between the common mode measurement and the differential measurement, the expression “differential measurement” referring to the measurement corresponding to the difference between the first electrical signal (i.e. reference signal or measured signal) and the second electrical signal (i.e., measured signal or reference signal) in the single earpiece embodiments and between the measurement signal and the reference signal in the dual earpiece embodiments. Such a correlation computation provides with an estimation of the impedance of the system and therefore of the quality of the measured signal.

There are many applications for the device 1a, 1b, particularly in medicine, such as monitoring patients suffering from neuronal diseases, e.g. epilepsy, screening and diagnosing neuronal diseases, screening and monitoring children suffering from attention disorders, sleep measurements, among others.

1. A device for detecting brain activity of a mammal, the device comprising at least one earpiece configured to be worn on an ear of the mammal, the earpiece comprising:
a main body comprising a housing portion and an elongation portion which extends along an elongation axis from the housing portion, the elongation portion being configured to be inserted into an ear canal of the ear of the mammal, and
an earmold configured to be inserted into an ear canal of the ear of the mammal, said earmold having a channel extending along a earmold axis, and a skirt surrounding the channel, the channel being adapted to receive said elongation portion to removably mount the earmold onto the main body, at least the skirt of the earmold being made of an elastically deformable material, said earmold comprising at least one electrode assembly, the electrode assembly comprising a measurement electrode arranged on an outer surface of the skirt of the earmold to contact the ear canal and configured to deliver an electrical signal representative of the brain activity of the mammal, said electrical signal having an electrical intensity;
a processing unit arranged in the housing portion of the main body,
said earpiece comprising at least one first electrical track arranged in said channel of the earmold and connected to the measurement electrode, and at least one second electrical track arranged in said elongation portion of the main body and configured to convey the electrical signal delivered by at least one measurement electrode to the processing unit, wherein the electrode assembly comprises a preamplifier having an intensity gain adapted to adapt the electrical intensity of the electrical signal delivered by the measurement electrode of said electrode assembly, the preamplifier being positioned upstream of the processing unit in a direction of propagation of the electrical signal delivered by the measurement electrode of said electrode assembly, so that the processing unit is configured to process the electrical signal adapted in intensity from the electrode assembly.

2. The device according to claim 1, wherein the preamplifier is adapted to amplify the electrical intensity of the electrical signal detected by the measurement electrode of the electrode assembly.

3. The device according to claim 1, wherein the preamplifier of the electrode assembly is housed in the elongation portion.

4. The device according to claim 1, wherein the preamplifier presents a working frequency range extending from 0.1 Hz to 1 kHz.

5. The device according to claim 1, wherein the preamplifier is coupled to an ESD circuit.

6. The device according to claim 1, wherein the preamplifier comprises a follower circuit.

7. The device according to claim 6, wherein the follower circuit is an active circuit.

8. The device according to claim 1, wherein the preamplifier comprises an impedance matching circuit.

9. The device according to claim 1, wherein the preamplifier is mounted on a printed circuit board of maximum dimension 3 mm.

10. The device according to claim 9, wherein said printed circuit board is made out of a flexible material, so as to be folded.

11. The device according to claim 9, wherein the preamplifier is adapted to amplify the electrical intensity of the electrical signal detected by the measurement electrode of the electrode assembly, and wherein said printed circuit board comprises a U-shape so as to embrace additional components embedded in the elongation portion.

12. The device according to claim 1, wherein the earpiece is further configured to transmit a sound signal into an ear canal by means of at least one electroacoustic transducer housed in the elongation portion, the preamplifier being attached to the electroacoustic transducer along a circumferential direction of the elongation axis.

13. The device according to claim 1, wherein the earmold is arranged to be rotatable about the elongation axis on the elongation portion so as to allow the measurement electrode to be directed to an area of the brain of said mammal, the second electrical track extending in a circumferential direction around the elongation axis on at least a portion of an outer surface of the elongation portion, the first electrical track presenting an end portion extending radially with respect to the elongation axis to be in contact with the second electrical track.

14. The device according to claim 1, wherein the housing portion is configured to be received in an ear conch of the ear of the mammal.

15. The device according to claim 1, wherein said at least one electrode assembly includes a plurality of electrode assemblies each comprising one measurement electrode and one preamplifier.

16. The device according to claim 15, wherein said plurality of electrode assemblies comprises at least first and second electrode assemblies, at least a first measurement electrode of the first electrode assembly being configured to deliver a first electrical signal and a second measurement electrode of the second electrode assembly being configured to detect a second electrical signal, said at least first electrical track comprises at least two first electrical tracks, and said at least second electrical track comprises at least two second electrical tracks, said at least two first electrical tracks being electrically insulated from each other,
said at least two second electrical tracks being electrically insulated from each other,
the processing unit being configured to detect brain activity as a function of said first electrical signal delivered by said first measurement electrode and adapted in intensity by a first preamplifier of the first electrode assembly and of said second electrical signal delivered by said second measurement electrode and adapted in intensity by a second preamplifier of the second electrode assembly, in particular as a function of a difference between said first electrical signal and said second electrical signal.

17. The device according to claim 16, wherein the first and second electrode assemblies are arranged on a single earpiece, the first and second measurement electrodes being spaced apart along the earmold axis, the two first electrical tracks being spaced apart along the earmold axis and the two second electrical tracks being spaced apart along the elongation axis.

18. The device according to claim 16, wherein the device comprises two earpieces configured to be arranged respectively in a first ear canal of the mammal and a second ear canal of the mammal and configured to convey respective electrical signals to the processing unit, and wherein the first and second electrode assemblies are arranged respectively on the two earpieces.

19. The device according to claim 1, wherein the processing unit is further configured to determine physiological or psychological state based on the detected brain activity.

20. A computer-implemented method for determining a physiological or psychological state of a mammal with the device according to claim 19, said method comprising:
determining an electroencephalogram signal of the mammal based on the delivered electrical signals,
determining an amplitude of at least one brain wave in a predefined frequency range as a function of said electroencephalogram signal, and
determining a psychic state based on said amplitude of at least one brain wave by comparing said amplitude to a predetermined threshold.

21. The method according to claim 20, further comprising computing a quality index of the electrical signals delivered by the measurement electrodes based on a correlation computation between a common mode measurement and a difference between said first electrical signal and said second electrical signal or a difference between two electrical signals.
